(19) United States
(12) Patent Application Publication
(10) Pub. No.: US 2025/0339020 A1
(43) Pub. Date: Nov. 6, 2025

(54) ENDOSCOPIC DEVICE, SYSTEM AND METHOD

(71) Applicant: The University Court of The University of Edinburgh, Edinburgh (GB)

(72) Inventors: Harry A C WOOD, Bath (GB); James M STONE, Bath (GB); Adam MARSHALL, Edinburgh (GB); Kevin DHALIWAL, Edinburgh (GB)

(73) Assignee: The University Court of The University of Edinburgh, Edinburgh (GB)

(21) Appl. No.: 18/862,426

(22) PCT Filed: Apr. 28, 2023
(86) PCT No.: PCT/GB2023/051131

§ 371 Date: Nov. 1, 2024

(30) Foreign Application Priority Data
May 4, 2022 (GB) 2206515.5

(51) Int. Cl. A61B 1/07; A61B 1/00; A61B 1/04; A61B 5/00

(52) U.S. Cl. CPC A61B 1/07; A61B 1/0011; A61B 1/043; A61B 5/0075

(57) ABSTRACT

An endoscopic device comprises a wall defining an interior space, a working channel in, or defined by, the interior space, and at least one optical fibre embedded or otherwise provided in the wall, wherein the endoscopic device has an outer diameter of less than 2.5 mm.

### INTRODUCTION

The present invention relates to an endoscopic device, system and method.

### BACKGROUND

It is known to provide medical endoscopic devices that incorporate optical biopsy fibres and working channels. The fibres serve to provide imaging or sensing data while arranged relative to a working channel such that during operation a medical device can be inserted through the working channel. Currently available devices generally have distal outer diameters greater than 2 millimetres.

There is a need for disposable, cost effective medical endoscopic devices with distal outer diameters at or below 2 millimetres with high flexibility to enable non-invasive and atraumatic access to parts of anatomy that are difficult to reach, such as the distal lung.

It is known to provide a bundle of miniaturised optical fibres that can be fed into a traditional bronchoscope, but without a working channel for solid objects such as diagnostic and treatment tools.

Olympus produces bronchoscopes that include an approximately 3 millimetre outer diameter device which uses peripheral optic devices such as lenses at the distal end.

JP2008200098A discloses a non-disposable optical coherence tomography (OCT) fibre provided in a sheath wall for use in biopsy procedures on small lumens. The OCT part of the sheath can be slid in and out.

U.S. Pat. No. 8,333,691 B2 describes a flexible multi-lumen catheter probe with multiple fibres and working channel, and separate illumination and imaging systems and fibres. The flexible catheter probe is a disposable component and an injection moulded component or an extruded component.

EP1948056A2 describes a catheter that accommodates an optical fibre probe for sensing the catheter tip's environment. The optical fibre has a distal end for illuminating tissue and receiving light energy from tissue.

U.S. Pat. No. 5,419,312 describes a multi-functional, multi-fibre endoscope.

U.S. Pat. No. 6,458,076B1 describes a multi-lumen flexible endoscope wherein the multi-lumen shaft has an outer diameter of 6 mm or less, and separate illumination fibres are used.

### SUMMARY

In a first aspect there is provided an endoscopic device comprising a wall defining an interior space, a working channel in, or defined by, the interior space, and at least one optical fibre embedded or otherwise provided in the wall, wherein the endoscopic device has an outer diameter of less than 2.5 mm, optionally less than or equal to 2 mm. The at least one optical fibre may comprise at least one optical imaging fibre and/or at least one optical sensing fibre.

The wall may comprise plastic material and/or heat-shrink material.

The plastic material and/or heat-shrink material may have been subject to a plastic processing method, optionally at least one of a heat-shrink process, a plastic welding process, an injection moulding process and/or an extrusion process. The embedding or otherwise providing the at least one optical fibre in the wall may comprise adhering the at least one optical fibre to at least part of the wall. The embedding may comprise embedding within material of the wall.

The heat shrink material may comprise a biocompatible material. The heat shrink material may comprise at least one of Pebax heatshrink, PET, FEP, or PTFE heat shrink material. The heat shrink material may form the outer surface of the device. The heatshrink material may be stripped or otherwise removed from the device after performance of the heat shrink process.

The at least one optical fibre may comprises optical fibre(s) configured both to provide illumination to a target region at a distal end of the fibre(s) and to receive light from the target region, and to transmit the received light from the distal end of the fibre(s) to a proximal end of the fibre.

The or each optical fibre may comprise an optical imaging fibre that has an outer diameter in a range 0.1 mm to 1.0 mm, optionally in a range 0.2 mm to 0.5 mm.

The or each optical fibre may comprise a sensing fibre, optionally a single core fluorescence sensing fibre, and may have an outer diameter in a range 0.01 mm to 1.0 mm, for example an outer diameter of 0.04 mm.

The optical fibre, or one or more or each of the optical fibres, may comprise a single core or may comprise a plurality of cores. A regular array of cores may be provided, surrounded and/or separated by cladding. The cores may comprise doped silica, for example fluorine-doped silica or germanium-doped silica. The cladding may comprise silica, for example pure silica.

The end-face of the or each optical fibre and/or core(s) of the fibres may be substantially flat and/or may not include optical components.

The at least one optical fibre may be embedded in the wall over an embedded length that comprises at least 50% or at least 80% of the length of the wall and/or at least 50% or at least 80% of the length of the optical fibre; and/or the at least one optical fibre may be embedded in the wall over an embedded length that is at least 15 cm, optionally at least 30 cm, optionally at least 50 cm.

Both the wall and the optical fibre may be flexible. The optical fibre may be embedded such that when flexed the wall and the optical fibre remain in contact over all of the embedded length.

The at least one optical fibre may comprise an optical fibre that consists of a bundle of fibres that are configured in combination to provide imaging and/or sensing.

The at least one optical fibre may be configured to perform at least one of widefield imaging, confocal imaging, and/or fluorescence imaging.

The at least one optical fibre may be arranged relative to the working channel such that in operation a region illuminated and/or imaged using the at least one optical fibre is accessible to a medical device inserted through the working channel.

The working channel may comprise a liner.

The liner may be formed of polytetrafluoroethylene (PTFE), polyimide, FEP or other polymer material.

The working channel may be configured to receive at least one medical device, optionally a therapeutic, surgical or diagnostic tool and/or a sensing device.

The working channel may have an inner diameter of less than 1.8 mm, optionally less than or equal to 1.5 mm, optionally less than or equal to 1.2 mm, optionally less than or equal to 1 mm.

The device may further comprise at least one sensing fibre that is also embedded or otherwise provided in the wall.

The at least one sensing fibre may comprises a fibre for use in performing spectroscopy, optionally Raman spectroscopy.

The wall may have has a thickness that varies, optionally that varies continuously, around its circumference.

The at least one optical fibre may be located in a thickest portion of the wall.

The thickest portion of the wall may comprise a half, third, quarter or eighth of the circumference for which the wall has the highest average thickness.

The device may be a disposable device, optionally a single-use disposable device.

The optical fibre may be connectable at a proximal end to an endoscopic imaging apparatus that includes a light source configured to input light to the at least one optical imaging fibre at the proximal end, and a light detector configured to detect light transmitted from the distal end to the proximal end of the at least one imaging fibre.

In a further aspect, which may be provided independently, there is provided a method of forming an endoscopic device, comprising inserting at least one optical fibre, optionally at least one optical imaging fibre, into a tube of material, and performing a process to at least partially embed or otherwise provide the at least one optical fibre in a wall of the tube.

The process may comprise at least one of a plastic processing method, a heat-shrink process, a plastic welding process, an injection moulding process and/or an extrusion process. The process may comprise adhering the optical fibre to at least part of the wall.

The method may further comprise providing a mandrel in the tube and removing the mandrel from the tube thereby forming a working channel within the device.

The mandrel may include a liner, and the removing of the mandrel may comprise leaving the liner within the tube to form a wall of the working channel.

The endoscopic device that is formed may comprise a device as claimed or described herein.

In a further aspect there is provided an endoscopic system comprising a device as claimed or described herein, and an endoscopic apparatus that includes a light source configured to input light to the at least one optical imaging fibre at the proximal end, and a light detector configured to detect light transmitted from the distal end to the proximal end of the at least one imaging fibre.

In another aspect there is provided a method of imaging a region of a subject comprising inserting a device as claimed or described herein into the subject, transmitting light from a light source through the at least one optical fibre to said region of the subject, and detecting at a proximal end of the at least one optical fibre light received from the region of the subject.

In another aspect there may be provided an endoscopic device with a working channel, an imaging channel, a possible multiplicity of optical fibres, that uses the same optical fibre for illumination and collection of light. The device may be configured to perform fluorescence endomicroscopy imaging and/or any suitable zero working distance imaging techniques, circumventing the need for distal optical devices. The optical fibre may be embedded into the wall of the working channel and the working channel can be used to insert a second medical device to access the area under investigation for diagnosis and treatment. The second medical device may be guided to the identified suspicious tissue region while maintaining positive identification of the region using the optical fibre. The device may be formed using a heat-shrinking process that embeds the optical fibre into the wall of the working channel, thus providing both flexibility and sub 2-millimetre outer diameter.

In another aspect there is provided an endoscopic probe providing targeted diagnosis and therapy to the distal lung. The probe may comprise an optical fibre for fluorescence endomicroscopy, configured to provide sufficient information to identify tissue regions of interest (for example cancerous lesions) and an adjoined working channel of 1.2 millimetres or less inner diameter. The tool may have an outer diameter of less than 2.5 millimetres, optionally 2 millimetres or less to enable non-invasive and atraumatic access to the distal lung. The use of fluorescence endomicroscopy may allow for zero-working-distance imaging techniques which circumvent the need for distal optics and electronics This may be achieved, for example, by using the same optical fibre for both illumination and collection of light, circumventing the need for distal optics and electronics, and the integration of both working channel and optics into a monolithic device.

The probe may provide, or be used in, a method of preliminary diagnosis of suspicious tissue regions for investigation; provide or be used in a method to guide additional diagnostic or therapeutic tools to the identified suspicious tissue region while maintaining positive identification using the diagnostic method. The method of encasing the fibres may use heat shrink lamination techniques and biocompatible polymers. The one or more optical fibres may be embedded in the wall due to the heat shrink lamination technique. The probe may be disposable and configured for single use, for example by using industrially available materials and methods. The provision of a single-use disposable flexible bronchoscope may help to avoid cross-contamination and increase resource utilisation.

In another aspect, there is provided a micro endoscopic probe providing targeted diagnosis and therapy to the distal lung. The probe may comprise an optical fibre for fluorescence endomicroscopy, an optical sensing fibre sensing for use in performing spectroscopy and an adjoined working channel of 1.2 millimetres or less inner diameter. The tool may have an outer diameter of 2 millimetres or less to enable non-invasive and atraumatic access to the distal lung. The wall of the channel may have a thickness that varies around its circumference. At least one of the optical fibres may be located in the thickest portion of the wall.

In another aspect there is provided a method of preliminary diagnosis of suspicious tissue regions for investigation, enabling access to hard-to-reach areas of the human body (such as the distal lung). Its construction may be such as to enable single-use disposability to increase clinical throughput and simplicity. A disposable and minimally invasive device capable of targeted diagnosis and therapy to the distal lung may be provided. The device and/or system may be configured to perform any one or more of multiple spectroscopic methods of tissue characterisation coupled with a working channel. The working channel may allow access to the same tissue site that is being imaged to other diagnosis or therapeutic tools, while maintaining positive identification.

The device may provide or be used in a diagnosis and therapeutic tool to provide cellular-resolution imaging in real-time for clinicians to carry out preliminary diagnoses and therapeutic procedures in vivo, for example speeding clinical workflow and minimising patient harm.

It is envisaged that the device could also be adapted to suit other organs and tissues by including other sensing or imaging fibres and devices, capillaries for fluid delivery and extraction, or micromechanical tools. Other organs may have stricter outer diameter requirements, but by omitting the imaging fibre in some embodiments the device may be made thinner than one millimetre, while for example still providing a full suite of chemically specific spectra or other desired spectroscopic functionality.

The method of encasing the fibres may use heat shrink lamination techniques to mould a biocompatible tube around an optical (imaging) fibre, and a removable mandrel and Polytetrafluoroethylene (PTFE) liner to form the working channel. The fibre may be encased in the polymer along its whole length. The heat shrink may be made of Pebax, Polyethylene terephthalate (PET), Fluorinated ethylene propylene (FEP), PET or other suitable materials.

Features in one aspect may be applied as features in any other aspect, in any appropriate combination. For example, features of any one or more of device, system, probe or method aspects may be applied as features in any one or more other of device, system, probe or method aspects.

### BRIEF DESCRIPTION OF THE DRAWINGS

Various embodiments will now be described by way of example only, and with reference to the accompanying drawings, of which:

FIG. 1 is an image of a device with a plastic splitter separating the imaging fibre and working channel;

FIG. 2 is an image of the distal head of the device with an optical imaging fibre integrated into its wall;

FIG. 3 is a schematic diagram showing the optical imaging fibre of the device connected to an imaging apparatus;

FIG. 4 is an image of the distal head of a probe with an optical imaging fibre and an optical sensing fibre integrated into its wall;

FIG. 5 is an image of the distal head of a probe with optical imaging fibre encased in high durometer Pebax; and

FIG. 6 is an image of a catheter with biopsy forceps emerging from the distal end of the working channel, and illumination from the distal end of the imaging fibre.

### DETAILED DESCRIPTION

A device according to an embodiment combines an optical biopsy diagnosis fibre, capable of identifying suspicious tissue region(s) or other region(s) of interest, with a working channel that enables access to the same region(s), while maintaining positive identification. The working channel can enable further additional diagnostic, therapeutic or other tools to be deployed in real-time to the suspicious tissue region identified. The device is flexible and has a sub 2.5 millimetre, optionally sub 2 millimetres diameter, enabling minimally invasive non-surgical access to hard-to-reach areas of the human body (such as the distal bronchioles) while minimising patient stress and providing highly targeted therapeutic and optical biopsies. In various embodiments, the optical imaging fibre has an outer diameter in the range of 0.1 to 1 millimetres, optionally in the range 0.2 to 0.5 millimetres.

The optical imaging fibre or the optical sensing fibre may comprise a single core surrounded by a cladding or an array of multiple cores surrounded by a cladding. The cores may be a regular array of germanium doped silica cores, surrounded and separated by pure silica glass cladding. In other embodiments, fluorine doped silica may be used for the cladding. The optical imaging fibre may be comprised of a bundle of fibres that are configured in combination to provide imaging. The optical sensing fibre may be comprised of a bundle of fibres that are configured in combination to provide sensing. Any suitable imaging, sensing and/or spectroscopy procedures may be performed using the fibre(s), for example fluorescence imaging and/or Raman spectroscopy.

The device makes use of a single imaging fibre without further optics (at the distal end) by using the same optical fibre for both illumination and collection of light. This enables the probe to have a smaller distal cross section size (less than 2 millimetres) and flexibility. The probe can provide an adequate method of preliminary diagnosis of suspicious tissue regions for investigation using an imaging fibre, and through its working channel provides a method to guide additional diagnostic or therapeutic tools to the identified suspicious tissue region while maintaining positive identification using the diagnostic method.

A method of forming devices in some embodiments, comprising encasing the fibres in biocompatible polymer along the whole fibre length, can confer device characteristics of strength, kink resistance and flexibility. The device structure is such that it may be sufficiently cost effective to allow disposal after a single use. The heat shrink may form the outer surface of the device.

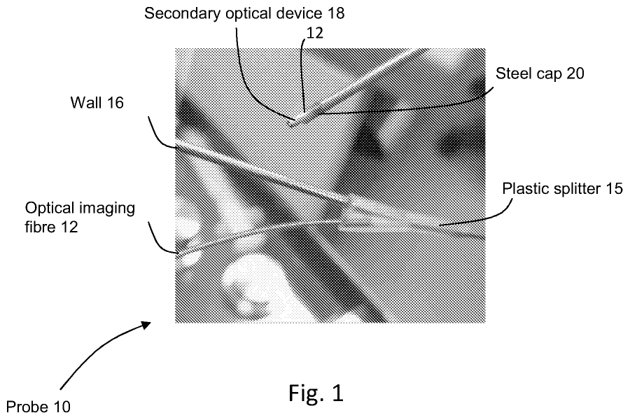

FIG. 1 is an image of a device in the form of a probe 10 according to an embodiment, showing an outer wall 16 of the device in an inner space of which a working channel 14 is provided, an optical imaging fibre 12 that is embedded in the wall 16, and a plastic splitter 15 that separates the imaging fibre 12 and wall 16 of the device at the proximal end of the device 10. The imaging fibre 12 is located in the wall 16 of the device 10 along the length of the device 10 all of the way from the splitter 15 to the distal end of the device 10. The splitter 15 helps protect the fibre 12 at the point where the fibre exits the wall 16 of the device, to reduce the chances of mechanical damage in use. In this embodiment, the distal end of the imaging fibre 12 is fixed to the distal end of the device using epoxy, but it is free inside the wall 16 along its length. In other embodiments, the epoxy may fix the imaging fibre 12 along all or most of the length of the device. Any other suitable adhesive can be used in place of epoxy in other embodiments.

In the embodiment shown in FIG. 1 a secondary optical device 18 has been inserted into the working channel 14. The secondary optical device 18 is shown in FIG. 1 emerging from the distal end of the working channel 14. A steel distal end cap can be provided at a distal end of the secondary optical device 18 inserted in the working channel. The device 10 can be capped with polyimide tubing and epoxy

It will be understood that in FIG. 1 the endoscopic device 10 has been bent around on itself so that the distal end and a proximal region can be shown in the same image

The space between the working channel 14 and around the optical imaging fibre 12 bounded by the wall 16 made of biocompatible polymer can, for example, be sealed by epoxy. In other embodiments, the epoxy can fill a length that is smaller than the length of the working channel 14 and/or or space between the working channel 14 and wall 16.

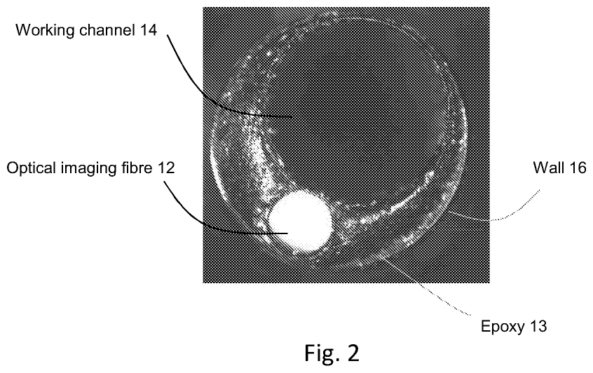

FIG. 2 is an end-on image of the distal head of the probe 10 of FIG. 1, showing the optical imaging fibre 12 embedded in the wall 16 of the probe. The probe 10 has a 2 millimetre outer diameter and the optical imaging fibre 12 has a 0.45 millimetre diameter. The inner diameter of the working channel 14 is 1.2 millimetres. The optical imaging fibre 12 and/or core of the fibres is substantially flat-faced at its distal end face and does not include any optical components. The embodiment shown includes an outer and inner layer of polyimide with the imaging fibre 12 between the two layers. The remaining space between the inner and outer layer of polyimide is filled with an epoxy 13 over at least part of the length of the working channel or wall. In other embodiments, the remaining space between the inner and outer layer of polyimide is partially filled with an epoxy. The inner layer of polyimide forms the boundary of the working channel 14. Thus, the probe 10 is formed of an outer layer of polyimide tubing, epoxy 13, the fibre 12, and an inner layer of polyimide that makes the working channel 14. The wall is formed of the combination of inner and outer layers of polyimide and the epoxy 13. The polyimide as used in this embodiment is not heat-shrinkable.

Any suitable therapeutic, surgical or diagnostic tool and/or a sensing device may be inserted into the working channel 14. In some embodiments, the working channel may, for example, have an inner diameter of less than 1.8 millimetres, optionally less than or equal to 1.5 millimetres, optionally less than or equal to 1.2 millimetres and optionally less than or equal to 1 millimetre. The working channel may, for example, have a minimum diameter of 0.5 mm. In the embodiment of FIG. 1, the working channel has a diameter of 1.2 mm, the imaging fibre has a diameter of 0.2 mm, and the probe 10 has a diameter of 2.0 mm.

In the device of FIG. 1, the optical imaging fibre was fabricated using two kinds of stock glass, namely undoped silica tubes and rods and germanium doped silica. The undoped silica tubes and rods were Superasil F300 (Heraeus), and the germanium doped silica was from OM1 preforms with a core to cladding ratio of 0.75, parabolic index profile, and 0.3 peak numerical aperture (Draka, Prysmian). Both materials are mass produced for the telecoms industry, allowing the devices to be sufficiently cost effective to be for single use and subsequently disposable. The optical imaging fibre was produced using known heating and drawing techniques to produce an imaging fibre including multiple cores. Any suitable optical fibre, either single core or multi-core and produced using any suitable techniques, can be used in other embodiments.

In use, the optical imaging fibre 12, and/or core(s) of the fibre(s), is connected to an optical setup at the proximal end and may be substantially flat at its end-face. The optical setup may include an endoscopic imaging apparatus that includes a light source configured to input light to the at least one optical imaging fibre 12 at the proximal end of the device. The light source may for example be a light emitting diode (LED). In other embodiments, the light source may be a laser. The imaging apparatus may also include a light detector configured to detect light transmitted from the distal end to the proximal end of the at least one imaging fibre. The optical fibre is configured to transmit light in both directions along its length. It provides illumination to a target at the distal end of the fibre. The optical fibre also receives light from the target region and transmits the light from the distal end of the fibre to the proximal end of the fibre.

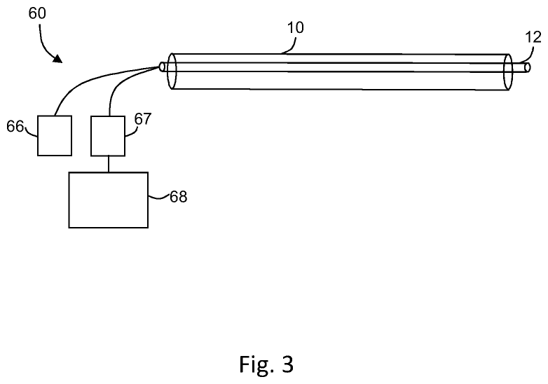

FIG. 3 is a schematic illustration of an imaging apparatus 60 that can be connected to the imaging fibre 12 in one mode of operation. The imaging apparatus 60 comprises a laser scanning unit 66, sensor unit 67 and processing apparatus 68. The laser scanning unit 66 is a proximal laser scanning unit linked to an interface with the multi-core optical fibre 12.

The optical fibre 12 and laser scanning unit 66 are configured to perform optical endomicroscopic imaging, also referred to as optical endomicroscopy (OEM) at the distal end of the fibre 12. In the present embodiment, the light source is a laser source with a wavelength less than or equal to 450 nm. In other embodiments, a light emitting diode (LED) with a wavelength less than or equal to 450 nm can be used. In embodiments where and LED is used as a light source, wide field illumination may be used for imaging. Although in the present embodiment images are obtained using optical endomicroscopy, in other embodiments any suitable method of obtaining images or other information may be used. In some embodiments, the device may be configured to perform Raman spectroscopy. For embodiments using Raman spectroscopy, a laser source with wavelength ranging from 532 nm to 785 nm and/or 850 nm to 1064 nm may be used. In some embodiments, wide field illumination may be used concurrently OEM or Raman spectroscopy. In some embodiments, a supercontinuum laser source may be used for the light source.

The imaging apparatus 60 is used to obtain images of the distal lung. In other embodiments, the endoscope device 10 may be used to position the optical fibre 12 in any suitable anatomical location in any human or animal subject. For example, the device 10 may be used to generate frame sequences or other images when navigating along the pulmonary tree, bronchioles, the urinary or gastrointestinal tract, or in laparoscopy, colonoscopy, biliary tree endoscopy, wireless capsule endoscopy or larynx endoscopy.

The laser scanning unit 66 is configured to send laser light down the optical fibre 12 into the distal lung. The light from the laser scanning unit 66 may generate auto-fluorescence of the lung and/or may cause fluorescence of fluorescent substances that have been introduced into the lung. In some embodiments, stains are added to the lung. The stains are configured to fluoresce when excited with appropriate light from the laser scanning unit 66.

Fluorescent light generated in the distal lung passes up the optical fibre and is received by the sensor unit 67. The sensor unit 67 converts the received light into electrical signals. The sensor unit houses collection optics as well as detectors. Collection optics can include one or more of filters, lenses, reformatters, and mirrors or any other suitable optical device. The detector can, for example, comprise a spectrometer, single photon detectors or charge-coupled device (CCD) chip. Single photon detectors in embodiments are usually implemented using avalanche diodes. The sensor unit 67 passes the electrical signals to processing apparatus 68. Processing apparatus 68 may comprise any appropriate computing device, for example, a desktop or laptop computer, or mobile device, or any suitable processor or combination of processors, or ASICS, FPGAs or other circuitry. In further embodiments, the laser scanning unit 66, the sensor unit 67 and processing apparatus 68 may be replaced by one combined apparatus. Functions described as being performed by the laser scanning unit 66 and the sensor unit 67 may be performed by the processing apparatus 18, or vice versa.

Processing apparatus 68 is configured to process the electrical signals received from the sensor unit 67 to obtain a frame sequence comprising a plurality of image frames. In the present embodiment, the electrical signals are representative of light received from individual cores of the fibre 12. The processing apparatus 68 combines signals from different cores to form each frame of the frame sequence. Processing apparatus 68 may obtain a sequence of image frames from which image effects caused by the individual cores of the fibre 12 have been removed. The frame sequence, or other images obtained using the optical imaging fibre 12, may be displayed in real time such that a region of the patient or other subject can be viewed whilst a procedure is performed on at least part of the region using a tool inserted through the working channel.

Any other suitable imaging apparatus or selected components thereof may be used in other embodiments. For example, imaging apparatus or components of such apparatus, for example light sources, detectors, optical components and/or processors, as described in any of WO2017/0158331, WO2017/174998, WO2017/149310, WO2018/007829, WO2017/203272, WO2018/134622, WO2018/203088,

WO2019/138220, WO2019/243760 or W02022/034055 as appropriate, each of which is incorporated herein by reference. The imaging apparatus may be configured to perform any suitable type of imaging, for instance widefield imaging, confocal imaging, and/or fluorescence imaging, for example using any suitable known techniques.

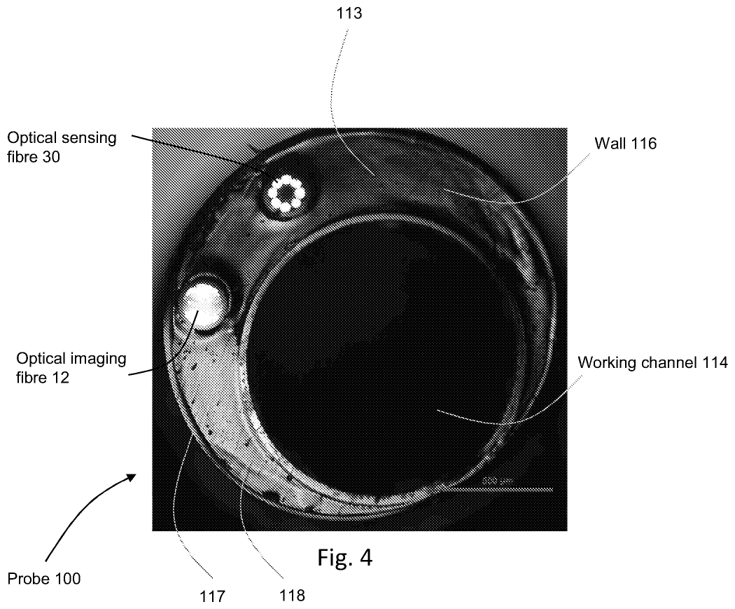

FIG. 4 is an image of the distal head of device 100 in a further embodiment. The device 100, in the form of an endoscopic probe, comprises an optical imaging fibre 12, for example as described in relation to FIG. 1, and a further optical sensing fibre 30, both of which are embedded in the wall 116 of the probe. A working channel 114 is also shown. The embodiment shown includes an outer layer of polyimide 117 and an inner layer of polyimide 118 with the imaging fibre 12 between the two layers. The remaining space between the inner and outer layers 117, 118 of polyimide is filled with an epoxy 113 over at least part of the length of the device. In other embodiments, the remaining space between the inner and outer layers 117, 118 of polyimide is partially filled with an epoxy 113. The inner layer of polyimide 118 forms the boundary of the working channel 114. The combination of the layers 117, 118 of polyimide and the epoxy 113 forms the wall 116.

In some variants, the wall 116 and the working channel 114 may have the same or similar properties as the wall 16 and working channel 14 of the embodiment of FIG. 1.

The further optical sensing fibre 30 is a fibre configured for use in Raman spectroscopy. Any suitable fibre may be used as an optical sensing fibre, for example a fibre or component(s) of a fibre as described in https://arxiv.org/abs/2012.08836 or https://onlinelibrary.wiley.com/doi/full/10.1002/jbio.202000488 may be used as the or a sensing fibre in embodiments. The further fibre 30 may for example be made using hollow capillaries in its structure, be multi core or single core, and can potentially use different dopants in its glass to those used in the imaging fibre 12 to change its optical properties (for example, fluorine for one of the fibres, germanium for the other of the fibres.

In other embodiments, the further optical sensing fibre(s) may be used for other modalities of imaging, such as fluorescence imaging, or any other sensing or measurement procedure. By embedding both the optical fibre and the further optical fibre in the wall of the device, a flexible device with known alignment and/or field-of-view of both optical fibre and further optical fibre may be provided, for example such that both the optical fibre and the further optical fibre may be used to image and/or perform measurements on the same or overlapping or related regions of a subject.

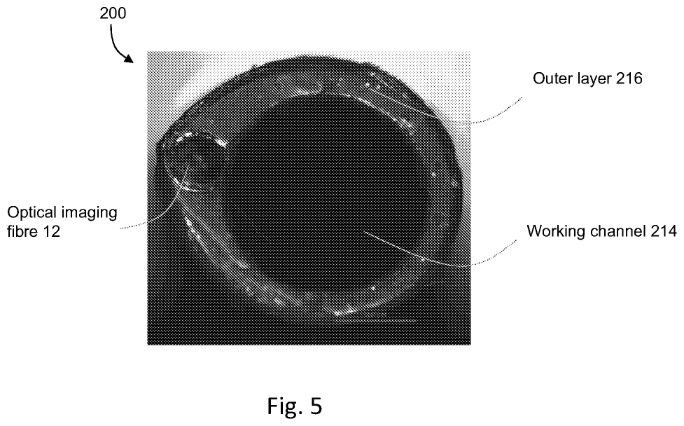

FIG. 5 is an image of the distal end of a device 200 according to another embodiment that comprises a PTFE-lined 1.2 millimetre diameter working channel 214 encased in 55 durometer Pebax. The outer layer 216 is made of high durometer thermoplastic Pebax heat-shrunk around the fibre 12 and working channel and liner. The diameter of the optical imaging fibre in this embodiment is 300 micrometres. A mandrel is used during the fabrication of this embodiment. The mandrel preserves the shape of the working channel during the fabrication of the device and is removed afterwards to create a working channel. No epoxy is used in the fabrication of this embodiment.

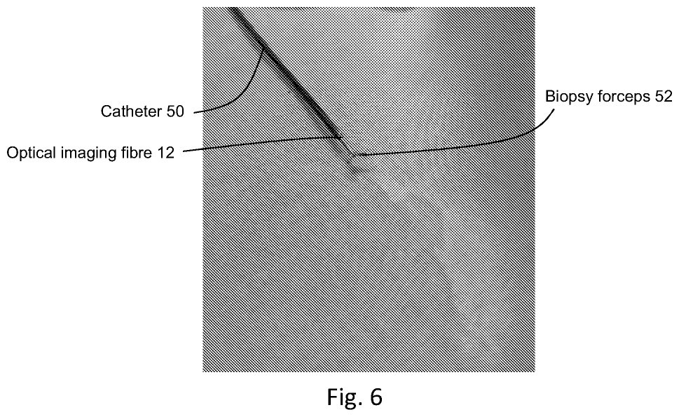

FIG. 6 is an image of a catheter 50 containing the endoscope probe 10 comprising an optical imaging fibre 12 and biopsy forceps 52 emerging from the distal head of the probe 10. In this embodiment the biopsy forceps 52 are made by Boston Scientific.

In the described embodiments, any suitable medical device, optionally any suitable therapeutic, surgical or diagnostic tool and/or a sensing device may be passed through the working channel to access a region of interest of the patient or other subject. The sensing device may for example comprise one or more imaging fibres in some embodiments.

According to some embodiments, a fabrication process of the device uses heat shrink lamination techniques to mould a biocompatible tube around an optical imaging fibre 12, thereby embedding the optical fibre in the wall 16, and a removable mandrel and PTFE liner to form the working channel 14. A heat gun may be used to thermally mould the heat-shrink at a temperature between 230 and 240 degrees centigrade. In other embodiments, the temperature used will be appropriate to the material properties of the thermoplastic material. The optical fibre is inserted into a tube of material, such as a biocompatible polymer for the process of embedding the fibre into the wall 16. A removable mandrel may be included in the tube before the heat shrinking process. Removing the mandrel defines an interior space which is called the working channel 14 and is has its inner surface covered with the PTFE liner. The PTFE liner, forms the wall 16 of the working channel 14. The devices use commercially available biocompatible materials for the heat shrinking method. Any of the embodiments of FIGS. 1 to 6 may, for example, be formed using such techniques.

Other embodiments may use catheter laminators, for example any suitable known catheter laminator, for the fabrication of the device.

Preferably, the mandrel has a circular cross-section and the working channel 14 produced by the removal of the mandrel is significantly cylindrical. In some embodiments, the completed probe 10 contains more than one optical fibre or more than one bundle of optical fibres.

The optical imaging fibre 12 may be of identical construction to the optical sensing fibre 30 or may be different in other embodiments. The optical imaging fibre 12 may consist of a single core while the optical sensing fibre 30 may consist of multiple cores and vice versa. The fibres may be identical or different in the material used for cladding, such as pure silica glass and fluorine doped silica glass. An optical sensing fibre may be used in place of an optical imaging fibre in some embodiments.

The working channel 14 may align with the central axis of the probe 10 causing the working channel 14 to be concentric with the probe 10 or it may be off the central axis resulting in a wall 16 that varies continuously in thickness along its circumference. In other embodiments, the thickness may vary in a non-continuous way along the circumference of the device. The thickest portion of the wall may comprise a half, third, quarter or eighth of the circumference for which the wall has the highest average thickness. The optical fibre may be located in a thickest portion of the wall.

The fibre is encased in the biocompatible polymer along its whole length. The liner may be formed of PTFE or other polymer material. The structure is small, strong, kink resistant and flexible. The heat shrink process involves applying heat to a heat-shrinkable biocompatible polymer until it contracts around the optical fibre(s), PTFE liner and optional removable mandrel In some variants, a second piece of biocompatible tube, under the heat shrink, and over the fibre and mandrel, is provided. When the heat shrink collapses it moulds this piece of tube to encase the fibre and mandrel, forming a monolithic shaft with the fibre and working channel embedded. For example, when the heat shrink is heated it squeezes and melts the middle layer tube (i.e. second piece of biocompatible tube) and moulds it around the fibre, encasing it and the liner and mandrel. The process results in the optical fibre being embedded into the wall 16 of the completed probe 10. The resulting device is flexible and contact is maintained between the wall 16 and the optical imaging fibre 12 when the device is flexed. At least one optical fibre is embedded in the wall 16 over a length that comprises at least 50% of the length of the wall and/or at least 80% of the length of the optical imaging fibre 12. The optical fibre may be embedded in the wall 16 over a length of at least 15 cm, optionally 30 cm or optionally at least 50 cm. The final device may be single-use disposable to avoid contamination.

Although heat shrink processes have been described for providing the optical fibres in a wall of the device, any other suitable processing method, for example any other suitable processing method can be used to provide the optical fibre(s) in a wall of the device, for example at least partially embedded in a wall of the device. For example, a plastic welding process, an injection moulding process, an extrusion process or any other suitable process may be used in place of the heatshrink process. In some embodiments, an extrusion process may be applied to the fibre or may be used to creating a hole which receives the fibre. In some embodiments, the fibre may be sandwiched between an inner tube and outer tube, with one of the tubes or both tubes together thus providing the or a wall.

It may be understood that the present invention has been described above purely by way of example, and that modifications of detail can be made within the scope of the invention.

Each feature disclosed in the description and (where appropriate) the claims and drawings may be provided independently or in any appropriate combination.

### References

JP2008200098A

U.S. Pat. No. 8,333,691B2

EP1948056A2

U.S. Pat. No. 5,419,312

U.S. Pat. No. 6,458,076B1

WO2017/0158331

WO20 17/174998

WO2017/149310

WO20 18/007829

WO20 17/203272

WO20 18/134622

WO20 18/203088

WO20 19/138220

WO20 19/243760

W02022/034055

https://arxiv.org/abs/2012.08836

https://onlinelibrary.wiley.com/doi/full/10.1002/jbio.202000488

https://proteus.ac.uk/technology/fibre-bundle/

https://link.springer.eom/article/10.1007/s00259-020-05021-4

https://www.olympus-europa.com/medical/rmt/media/en/Content/Content-MSD/Documents/Brochures/SRP-Periphery/E0428337EN_EEIII_BF-MP190_A4_EN.pdf

https://erj.ersjournals.eom/content/32/2/465 (“Novel thin bronchoscope with a 1.7-mm working channel for peripheral pulmonary lesions”)

1. An endoscopic device comprising a wall defining an interior space, a working channel in, or defined by, the interior space, and at least one optical fibre, optionally at least one optical imaging fibre, embedded or otherwise provided in the wall, wherein the endoscopic device has an outer diameter of less than 2.5 mm, optionally less than or equal to 2 mm.

2. The device according to claim 1, wherein the wall comprises plastic material and/or heat-shrink material.

3. The device according to claim 2, wherein the plastic material and/or heat-shrink material has been subject to plastic processing method, optionally at least one of a heatshrink process, a plastic welding process, an injection moulding process and/or an extrusion process.

4. The device according to claim 1, wherein the at least one optical fibre comprises optical imaging fibre(s) configured both to provide illumination to a target region at a distal end of the fibre(s) and to receive light from the target region, and to transmit the received light from the distal end of the fibre(s) to a proximal end of the fibre.

5. The device according to claim 1, wherein the or each optical fibre has an outer diameter in a range 0.1 mm to 1.0 mm, optionally in a range 0.2 mm to 0.5 mm.

6. The device according to claim 1, wherein the end-face of the or each optical fibre and/or core(s) of the fibres are substantially flat and/or do not include optical components.

7. The device according to claim 1, wherein the at least one optical fibre is embedded in the wall over an embedded length that comprises at least 50% or at least 80% of the length of the wall and/or at least 50% or at least 80% of the length of the optical fibre; and/or the at least one optical fibre is embedded in the wall over an embedded length that is at least 15 cm, optionally at least 30 cm, optionally at least 50 cm.

8. The device according to claim 7, wherein both the wall and the optical fibre are flexible, and the optical fibre is embedded such that when flexed the wall and the optical fibre remain in contact over all of the embedded length.

9. The device according to claim 1, wherein the at least one optical fibre comprises an imaging fibre that consists of a bundle of fibres that are configured in combination to provide imaging.

10. The device according to claim 1, wherein the at least one optical fibre is configured to perform at least one of widefield imaging, confocal imaging, and/or fluorescence imaging.

11. The device according to claim 1, wherein the at least one optical fibre is arranged relative to the working channel such that in operation a region illuminated and/or imaged using the at least one optical fibre is accessible to a medical device inserted through the working channel.

12. The device according to claim 1, wherein the working channel comprises a liner.

13. The device according to claim 12, wherein the liner is formed of polytetrafluoroethylene (PTFE), polyimide, FEP or other polymer material.

14. The device according to claim 1, wherein the working channel is configured to receive at least one medical device, optionally a therapeutic, surgical or diagnostic tool and/or a sensing device.

15. The device according to claim 1, wherein the working channel has an inner diameter of less than 1.8 mm, optionally less than or equal to 1.5 mm, optionally less than or equal to 1.2 mm, optionally less than or equal to 1 mm.

16. The device according to claim 1, further comprising at least one sensing fibre that is also embedded or otherwise provided in the wall.

17. The device according to claim 16, wherein the at least one sensing fibre, comprises a fibre for use in performing spectroscopy, optionally Raman spectroscopy.

18. The device according to claim 1, wherein the wall has a thickness that varies, optionally that varies continuously, around its circumference.

19. The device according to claim 18, wherein the at least one optical fibre is located in a thickest portion of the wall.

20. The device according to claim 1, wherein the device is a disposable device, optionally a single-use disposable device.

21. The device according to claim 1, configured such that the optical fibre is connectable at a proximal end to an endoscopic imaging apparatus that includes a light source configured to input light to the at least one optical fibre at the proximal end, and a light detector configured to detect light transmitted from the distal end to the proximal end of the at least one optical fibre.

22. The method of forming an endoscopic device, comprising inserting at least one optical fibre, optionally at least one optical imaging fibre, into a tube of material, and performing a process to at least partially embed or otherwise provide the at least one optical fibre in a wall of the tube.

23. The method according to claim 22, wherein the process comprises at least one of a plastic processing method, a heat-shrink process, a plastic welding process, an injection moulding process and/or an extrusion process.

24. The method according to claim 22, wherein the method further comprises providing a mandrel in the tube and removing the mandrel from the tube thereby forming a working channel within the device.

25. The method according to claim 24, wherein the mandrel includes a liner, and the removing of the mandrel comprises leaving the liner within the tube to form a wall of the working channel.

26. The method according to claim 25, wherein the endoscopic device comprises a device according to claim 1.

27. An endoscopic system comprising a device according to claim 1, and an endoscopic apparatus that includes a light source configured to input light to the at least one optical imaging fibre at the proximal end, and a light detector configured to detect light transmitted from the distal end to the proximal end of the at least one imaging fibre.

28. The method of imaging a region of a subject comprising inserting a device according to claim 1 into the subject, transmitting light from a light source through the at least one optical fibre to said region of the subject, and detecting at a proximal end of the at least one optical fibre light received from the region of the subject.
